(19) United States
(12) Patent Application Publication
(10) Pub. No.: US 2003/0228596 A1
(43) Pub. Date: Dec. 11, 2003

(54) Template-driven nucleic acid amplifications

(76) Inventors: Qiang Liu, Upland, CA (US); Piotr M. Swiderski, San Dimas, CA (US); Steve S. Sommer, Duarte, CA (US)

Correspondence Address: ROTHWELL, FIGG, ERNST & MANBECK, P.C., 1425 K STREET, N.W., SUITE 800, WASHINGTON, DC, 20005, US

(21) Appl. No.: 10/387,324

(22) Filed: Mar. 13, 2003

Related U.S. Application Data
(60) Provisional application No. 60/363,844, filed on Mar. 14, 2002.

(51) Int. Cl. C12Q 1/68; C12P 19/34

(52) U.S. Cl. 435/6; 435/91.2

(57) ABSTRACT

Truncated amplification (TA) provides high fidelity, template driven amplification of target nucleic acid molecules in which truncating oligonucleotides are used as primers to produce truncated terminal products that are in most, if not all, cases no more than three rounds of replication from the original template. In certain embodiments, TA can amplify target nucleic acids quadratically or geometrically, depending on the number of truncating oligonucleotides used for amplification.

### FIELD OF THE INVENTION

The present invention relates to nucleic acid amplification. More particularly, the present invention relates to methods for high fidelity amplification in which the original nucleic acid template has greater influence on the amplification process and products.

### BACKGROUND OF THE INVENTION

Amplification of nucleic acids is a fundamental tool of molecular medicine. A number of methods for enzymatic nucleic acid amplification in vitro have been developed, such as polymerase chain reaction (PCR) (Saiki, R. K. et al., 1985), ligase chain reaction (LCR) (Barany, F., 1991; Landegren, U. et al., 1988), rolling circle amplification (RCA) (Baner, J. et al., 1998; Lizardi P. M. et al., 1998), Qβ replicase (Kramer, F. R. et al., 1989), self-sustained sequence replication (3SR) (Guatelli, J. C. et al., 1990), and pyrophosphorolysis activated polymerization (PAP) (Liu, Q. et al., 2000). PCR, LCR, RCA and PAP have been adapted for allele-specific amplification (Sommer et al. 1989, Newton et al. 1989, Nichols et al. 1989, Wu et al., 1989, Landegren et al. 1988, Barany 1991, Lizardi et al. 1998, Liu and Sommer 2000).

PCR is the most popular method of nucleic acid amplification. PCR is an exponential process, in which the extension products from one cycle of amplification are used as templates for subsequent cycles of amplification. LCR and PAP can amplify exponentially, while RCA can amplify hyperbranchingly (super-exponentially).

PCR can achieve an amplification of ten million fold and is a valuable tool for making qualitative measurements. However, the exponential nature of amplification creates certain challenges. The polymerase error can be exponentially propagated in amplified products. The error rate of conventional PCR is problematic when amplifying from single cells or amplifying segments for protein functional analysis by in vitro translation. Allele-dropout and polymerase error are significant problems in single cell amplifications. In addition, the sensitivity of allele-specific PCR for detecting rare mutations is limited by the exponential amplification of any mismatch extensions. PCR is described in U.S. Pat. Nos. 4,683,202 and 5,468,613, fully incorporated herein by reference.

The present invention seeks to overcome at least some of the above problems associated with PCR. It is an object of the present invention to provide an amplification method in which polymerase errors are not propagated efficiently. It is another object of the invention to provide a method in which an original nucleic acid template exerts greater influence on the amplification process and products obtained therefrom. A further object of the invention thus is to provide an amplification method that is template-driven, thereby increasing the frequency of error-free products during nucleic acid amplification. Another object of the present invention is to provide chimeric oligonucleotides for achieving such template-driven amplification. These and other objects and advantages of the present invention, as well as additional inventive features, will be apparent from the following detailed description.

### SUMMARY OF THE INVENTION

The present invention provides a truncated amplification (TA) method for achieving high fidelity nucleic acid amplification. The present invention utilizes oligonucleotide primers and thermal cycling, but differs from PCR. TA amplifies non-exponentially using one or more truncating oligonucleotides and produces truncated terminal products that in most, if not all, cases are no more than three rounds of replication from the original template.

TA may be used to amplify single-stranded, as well as double-stranded, nucleic acids, preferably DNA or cDNA.

In one embodiment, TA generates truncated terminal products that accumulate geometrically (TA-geometric or TA-g), while in another embodiment, accumulation occurs quadratically (TA-quadratic or TA-q), depending on whether one or two truncating oligonucleotides, respectively, are used to amplify a target nucleic acid.

In a preferred embodiment, the truncating oligonucleotide(s) may be chimeric oligonucleotides, as defined below.

As shown herein, TA is useful when template-driven amplification of nucleic acids is desired to increase the frequency of error-free products.

### BRIEF DESCRIPTION OF THE FIGURES

FIG. 1A is a schematic diagram of TA-quadratic with two chimeric primers (P^{C} and Q^{C})

FIG. 1B is a detail of a truncation event.

FIG. 1C is a flowchart of TA-q product accumulation.

FIG. 2A is a schematic diagram of TA-geometric with one chimeric primer (P^{C})

FIG. 2B is a flowchart of TA-g product accumulation.

FIGS. 3A and B are graphs showing TA product accumulations with cycling by TA-q (A) and TA-g (B).

FIG. 4A is a schematic diagram relating to TA of the p53 gene.

FIG. 4B is an autoradiogram of a nondenaturing gel relating to TA of the p53 gene.

FIG. 4C is an autoradiogram of a denaturing gel relating to TA of the p53 gene.

FIG. 5 is a sequence analysis comparing PCR with TA-q and TA-g.

FIG. 6A is a schematic model relating to TA with different DNA polymerases.

FIG. 6B is an autoradiogram of a nondenaturing gel relating to TA of the p53 gene.

FIG. 6C is an autoradiogram of a denaturing gel relating to TA of the p53 gene.

FIG. 7A is a schematic model relating to TA adapted to allele-specific amplification.

FIG. 7B is an autoradiogram of a nondenaturing gel relating to TA adapted to allele-specific amplification.

FIG. 7C is an autoradiogram of a denaturing gel relating to TA adapted to allele-specific amplification.

### DETAILED DESCRIPTION OF THE INVENTION

Truncated amplification (TA) according to the present invention amplifies non-exponentially with truncating oligonucleotides as primers and produces truncated terminal products that in most, if not all, cases are no more than three rounds of replication from the original target transcript.

As used herein, the following words have the meaning set forth:

“Oligonucleotide” refers to a nucleic acid or nucleic acid analogue (or combination thereof), which can be derived from natural sources or synthesized chemically. It is generally used as a primer or a probe. Its exact size will depend on many factors, which in turn depend on the ultimate function or use of the oligonucleotide. An “oligonucleotide” used as a primer in accordance with the invention is generally the same as those primers used to perform amplification processes known in the art such as PCR. Examples of “oligonucleotides” used as primers are P and Q shown in the figures and Table 1.

“Primer” refers to an oligonucleotide which is used to initiate nucleic acid synthesis by a template dependent polymerase such as a DNA polymerase. The primer is complementary to a portion of a template nucleic acid. The primer must be sufficiently long to prime the synthesis of extension products when placed under conditions in which synthesis of a primer extension product which is complementary to a nucleic acid strand is induced, i.e., in the presence of four different nucleoside triphosphates and an agent for polymerization such as DNA polymerase in an appropriate buffer and at a suitable temperature. Table 1 shows examples of primers having lengths of about 25 nucleotides. The primers herein are selected to be at least substantially complementary to the different strands of each specific sequence to be amplified. This means that the primers must be sufficiently complementary to hybridize with their respective strands. Therefore, the primer sequence need not reflect the exact sequence of the template. Typically, the primers have exact complementarity to obtain the best detection results.

“Primer extension” refers to the process of elongation of a primer on a nucleic acid template. Using appropriate buffers, pH, salts and nucleoside triphosphates, a template dependent polymerase such as a DNA polymerase incorporates a nucleotide complementary to the template strand on the 3′ end of a primer which is annealed to a template. The polymerase will thus synthesize a faithful complementary copy of the template.

“Target nucleic acid” may be DNA or RNA, single or double-stranded, and present in a relatively pure form in the sample or one component of a mixture in the sample. In the case of RNA it is often useful to convert the RNA into cDNA using a reverse transcription step. The DNA or cDNA may be amplified directly according to the invention or subjected first to an initial amplification, such as by PCR.

A “truncating oligonucleotide” means an oligonucleotide primer that can be extended by a polymerase such as DNA polymerase, and as a template can truncate or block primer extension, thereby producing a truncated extension product.

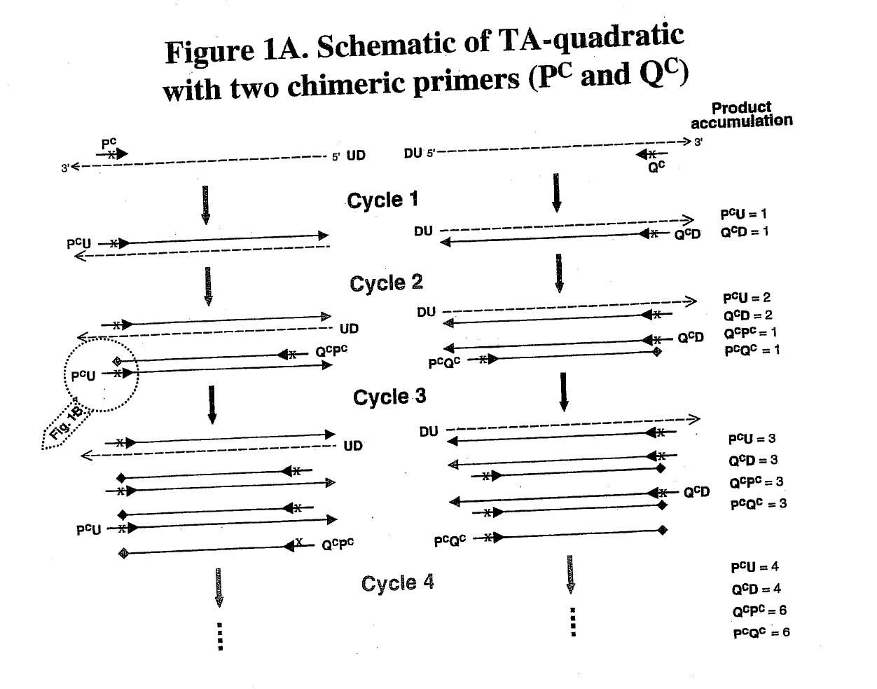

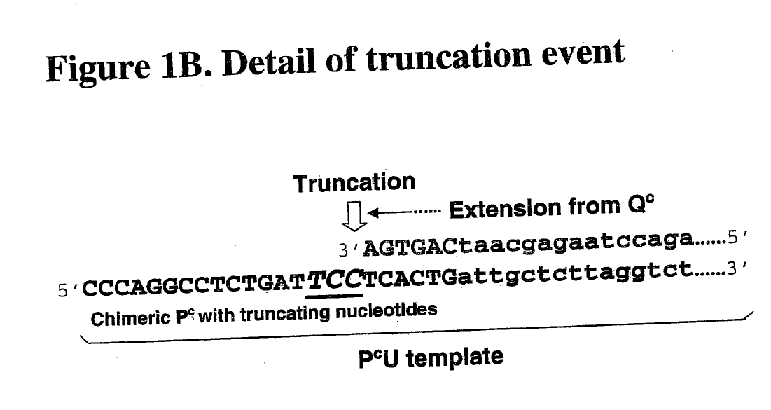

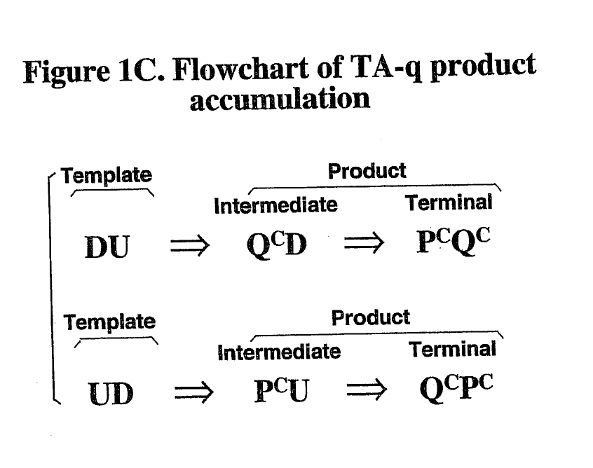

A “truncated extension product” or “terminal product” means a primer extension product, the synthesis of which is truncated or blocked as a result of a truncating oligonucleotide in the template strand. An example is shown in FIG. 1B. The truncated extension product or terminal product generally should not function as a template for further amplification, although exceptions may occur and are within the scope of the present invention. In a preferred embodiment, the truncated extension product or terminal product is sufficiently truncated or blocked such that it does not function as a template for further amplification.

A “chimeric oligonucleotide” or “truncating chimeric oligonucleotide” means a truncating oligonucleotide that contains one or more truncating nucleotides. Examples (p^{C} and Q^{C}) are shown in the figures and Table 1.

“Truncating nucleotides” means nucleotides or nucleotide analogues in a truncating oligonucleotide collectively through which a polymerase is at least sometimes, and preferably mostly always, unable to read, thereby producing a truncated extension product relative to the template strand. An example (“TCC”) is shown in FIG. 1B.

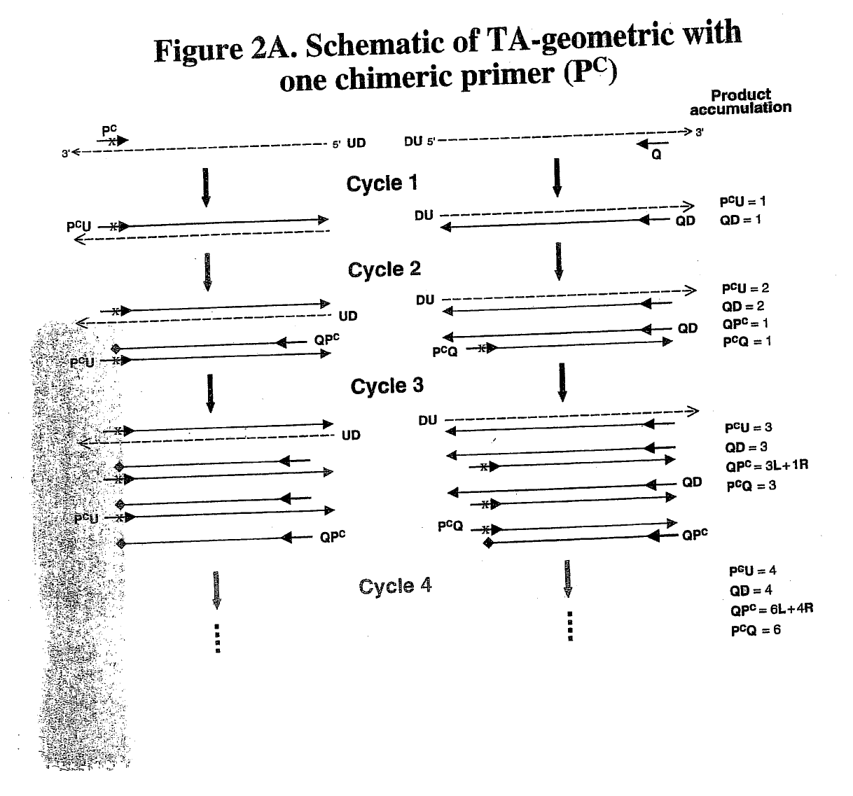

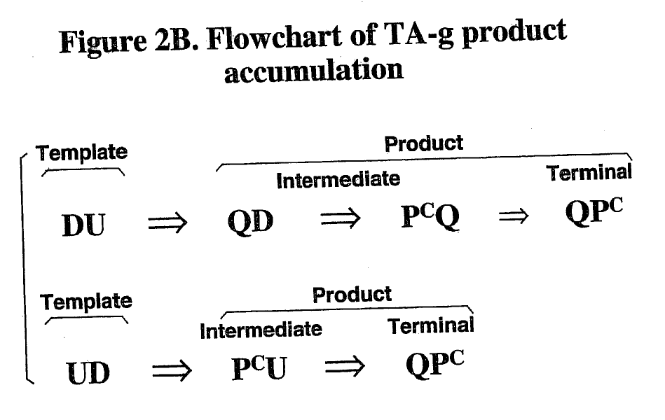

A “non-truncated extension product” or “intermediate extension product” or “intermediate, non-truncated extension product” is not truncated and can be used as a template for further amplification. For example, in TA-q, such extension product is produced in the first round of replication from the original template, while in TA-g, it is produced in the first and second rounds of replications from the original template. Examples of such extension products in FIG. 1 are P^{C}U and Q^{C}D, and in FIG. 2 are P^{C}U, QD and p^{C}Q.

An “upstream-directed original template” is exemplified by the template strand UD in FIG. 1A.

A “downstream-directed original template” is exemplified by the template strand DU in FIG. 1A.

An “upstream-directed extension product” or “upstream-directed, intermediate extension product” is exemplified by QCD in FIG. 1A.

A “downstream-directed extension product” or “downstream-directed, intermediate extension product” is exemplified by P^{C}U in FIG. 1A.

“Detectable amplification of the target nucleic acid” means detection of an amplified product by any detection means. Amplified products are typically detected as bands produced during electrophoresis, as shown in the figures.

In one embodiment, a method is provided for producing a truncated nucleic acid during an amplification reaction. The method comprises (A) treating a single strand template with a truncating oligonucleotide as a primer, along with nucleoside triphosphates and a polymerase under suitable conditions, such that the primer hybridizes to the original template and an intermediate extension product of the primer is synthesized which is complementary to the original template. The truncating oligonucleotide can block subsequent replication of the primer-containing strand, thereby producing a truncated extension product. The method also comprises (B) treating the sample under denaturing conditions, preferably heat denaturation, to separate the extension product from the original template; (C) treating the intermediate extension product with an oligonucleotide or a truncating oligonucleotide as a primer, such that the primer hybridizes to the intermediate extension product and the truncated extension product referred to above can be synthesized which is complementary to the intermediate extension product; and (D) optionally treating the sample under denaturing conditions to separate the extension product from the truncated extension product.

In another embodiment, a method is provided for amplifying a target nucleic acid contained in a sample. The method comprises (A) treating the nucleic acid with a truncating oligonucleotide as a primer, along with nucleoside triphosphates and a polymerase under suitable conditions, such that the primer hybridizes to the nucleic acid and an intermediate extension product of the primer is synthesized which is complementary to the nucleic acid; (B) treating the sample under denaturing conditions to separate the extension product from its template; (C) treating the original template with a truncating oligonucleotide as a primer, along with nucleoside triphosphates and a polymerase under suitable conditions, such that the primer hybridizes thereto and an intermediate extension product of the primer is synthesized which is complementary with the original template; (D) treating the intermediate extension product with an oligonucleotide or a truncating oligonucleotide as a primer, along with nucleoside triphosphates and a polymerase under suitable conditions, such that the primer hybridizes thereto and an extension product of the primer is synthesized which is complementary to the intermediate extension product, wherein extension of the primer can be truncated, thereby resulting in a truncated extension product; (E) treating the sample again under denaturing conditions to separate each of the extension products from its template; and (F) repeating steps (C) to (E) a sufficient number of times to result in detectable amplification of the target nucleic acid.

The detectable amplification corresponds to a measure of the truncated extension products and/or intermediate extension products produced during the amplification process, as shown in the figures.

In one embodiment, the target nucleic acid may be double-stranded and wherein its strands are separated by denaturation before or during step (A). The target nucleic acid also may be single-stranded.

In another embodiment, the target nucleic acid is DNA or cDNA, and the polymerase is a DNA polymerase, preferably Pfuturbo. In particular, with Pfuturbo, the chimeric oligonucleotides produced in accordance with a preferred embodiment of the invention were found to completely truncate polymerase elongation. The resulting truncated terminal products thereby were unable to serve as templates for further amplification due to the short length of the 3′ complementary region.

The steps of the methods of the invention may be conducted sequentially or simultaneously, or partially sequentially or partially simultaneously. A sufficient number of amplification cycles is preferably at least 5 cycles, and more preferably more than 15 cycles if the sample contains genomic DNA, preferably human genomic DNA.

In one embodiment, the truncating oligonucleotide is a chimeric oligonucleotide, as defined above.

In a preferred embodiment, the chimeric oligonucleotide comprises one or more, and preferably three, 3′-5′ reversed-deoxynucleotides. In a more preferred embodiment, the 3′-5′ reversed-deoxynucleotides are located about 6 to 8 nucleotides from the 3′ terminus of the oligonucleotide.

In another preferred embodiment, the chimeric oligonucleotide comprises one or more, and preferably three, 2′-OMe-ribonucleotides. In a more preferred embodiment, the 2′-OMe-ribonucleotides are located about 6 to 8 nucleotides from the 3′ terminus of the oligonucleotide.

The above chimeric oligonucleotides retain sequence specificity and primer extension activity. Preferred molar concentrations of chimeric and unmodified oligonucleotides are shown in Table 1 and preferably are about 0.05 μM to about 2 μM. Other possible molar concentrations for use in the invention can be determined by persons skilled in the art without undue experimentation.

In one embodiment, the chimeric oligonucleotide may be a polyamide nucleic acid (PNA)-DNA chimeric oligonucleotide. PNA-DNA chimeric oligonucleotides lack a phosphate backbone and are known to function as primers for polymerase-based reactions catalyzed by DNA polymerases. (Misra, H. S. et al., 1998). In accordance with this embodiment of the invention, template strands incorporating PNA-DNA chimeric oligonucleotides as part of the strand may potentially disrupt synthesis and replication by all DNA polymerases, thereby allowing polymerases lacking 3′ to 5′ exonuclease activity (e.g., Taq polymerase) to be more efficiently utilized.

The amplified and truncated nucleic acids produced in accordance with the invention may be detected as bands produced during gel electrophoresis (e.g., using staining and/or labeling techniques known in the art), as shown in the figures.

The invention also provides the use of one or more sequence-specific oligonucleotide probes, preferably labeled (radioactive or otherwise), capable of hybridizing with the amplified and truncated nucleic acid only if the sequence of the probe is complementary to a region of the amplified sequence. In another embodiment, a probe may be incorporated into the amplified sequence. Preferred probes include fluorescently labeled and other non-radioactive labeled probes.

The present invention also provides the above-described chimeric oligonucleotides, which are useful as primers in accordance with the present invention.

As stated above, TA generates truncated terminal products that may accumulate in a geometric (TA-g) or quadratic (TA-q) manner, depending on whether one or two truncating oligonucleotides, respectively, are used to amplify a target nucleic acid.

It is also recognized that one embodiment of the invention provides TA using two or more pairs of oligonucleotide primers, which is referred to as multiplex TA. In multiplex TA, for example, two or more different target nucleic acids in a sample may be amplified, simultaneously or otherwise, using TA-quadratic or TA-geometric alone or in combination to amplify the target nucleic acids.

### TA-quadratic (TA-q)

In one embodiment, TA-q comprises (A) treating a sample containing a target nucleic acid under denaturing conditions to produce original single strand templates from the target nucleic acid; (B) treating the original templates with a truncating oligonucleotide as a primer, along with nucleoside triphosphates and a polymerase under suitable conditions, such that the primers hybridize to a portion of the original templates, respectively, and an intermediate extension product of the primers is synthesized which is complementary to the original template; (C) treating the sample again under denaturing conditions to separate the extension products from the original templates; (D) treating both the intermediate extension products and the original templates with a truncating oligonucleotide as a primer, along with nucleoside triphosphates and a polymerase under suitable conditions, such that the primers hybridize to a portion of the intermediate extension products and the original templates, respectively, and an extension product of the primers is synthesized which is complementary to the intermediate extension products and the original templates, respectively, wherein extension of the primer on the intermediate extension products can be truncated, thereby producing a truncated extension product; (E) treating the sample again under denaturing conditions to separate the primer extension products from their templates; and (F) repeating, if necessary, steps (D) and (E) a sufficient number of times to result in detectable amplification of the target nucleic acid.

The detectable amplification corresponds to a measure of the truncated extension products and/or intermediate extension products produced, as shown in the figures.

The above embodiment is illustrated by the following preferred embodiment. However, the invention is not limited to the use of the chimeric oligonucleotides described below, but instead may be practiced with other types of truncating oligonucleotides, as defined herein.

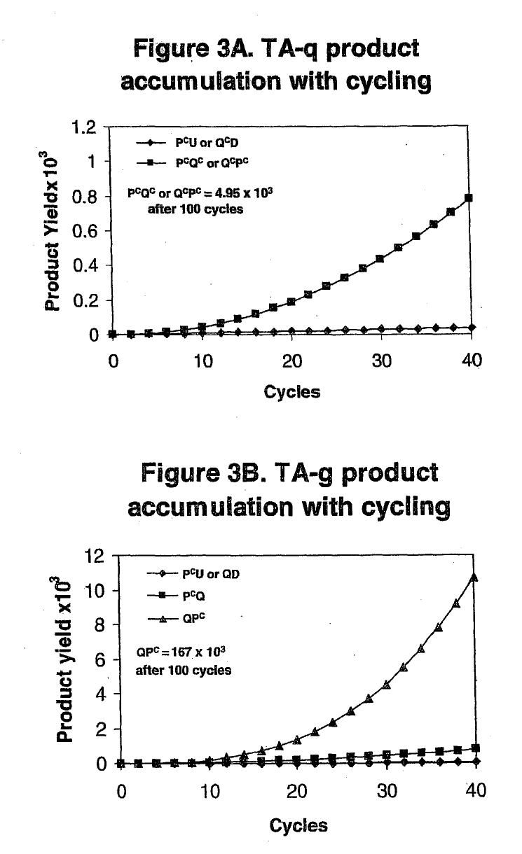

In a preferred embodiment, TA-quadratic (TA-q) utilizes two chimeric oligonucleotides, P^{C} and Q^{C} (Table 1). P^{C} and Q^{C} have 3′-5′ reversed deoxyribonucleotides (creating 5′ to 5′ and 3′ to 3′ linkages) or 2′ OMe-ribonucleotides at 6 to 8 nucleotides from the 3′ terminus. The two original template strands produce four types of intermediate extension products and terminal products (FIG. 1A). The extension products are terminal after two cycles of replication from the original template. In the first cycle, the truncating chimeric oligonucleotides, P^{C} and Q^{C}, are hybridized with the denatured single strand templates UD and DU (each 270 bases in the model system utilized), respectively, and extended to generate the intermediate extension products P^{C}U and Q^{C}D (235 and 221 bases) In the second cycle, Q^{C} and P^{C} are hybridized with the templates P^{C}U and Q^{C}D, respectively. They are extended to generate the truncated extension products Q^{C}P^{C} and P^{C}Q^{C} (each 170 bases) because the polymerase is blocked by the nucleotide analogues of the templated P^{C}U and Q^{C}D. Q^{C}P^{C} and P^{C}Q^{C} are terminal products that cannot be used as templates for further amplification, because they only have 6 nucleotides complementary to P^{C} and Q^{C} The products of TA-q accumulate approximately proportionally to the square of the number of cycles (FIG. 3A).

FIG. 1A shows a schematic diagram of TA-quadratic with two chimeric primers (P^{C} and Q^{C}) The downstream and upstream strands of the original template are denoted as DU and UD (each 270 bases in the model system), respectively. The truncating chimeric oligonucleotides are P^{C} and Q^{C}. They are complementary to UD and DU strands, respectively. TA-q is performed with both P^{C} and Q^{C} chimeric oligonucleotides. Segments P^{C}U (235 bases) and Q^{C}D (221 bases) are the intermediate, non-truncated extension products. Segments P^{C}Q^{C} and Q^{C}P^{C} (each 170 bases) are the terminal products and first appear in cycle 2. The maximum product accumulations are also shown on the right for each of the first four cycles. By cycle 4, P^{C}U=4, Q^{C}D=4, P^{C}Q^{C}=6 and Q^{C}P^{C}=6. By 40 cycles, P^{C}U or Q^{C}D=40, and P^{C}Q^{C} or Q^{C}P^{C}=780. The circled region is detailed in FIG. 1B.

FIG. 1B shows in detail a truncation event. The segment P^{C}U is generated in the first cycle by extending the chimeric oligonucleotide PC with the three truncating nucleotides underlined. In the second cycle, chimeric oligonucleotide Q^{C} is annealed with P^{C}U as template and is extended. The product Q^{C}P^{C} is truncated because the polymerase is unable to read through the modified nucleotides on the template. The truncated product Q^{C}P^{C} cannot be used as template for subsequent amplification.

FIG. 1C shows a flowchart of TA-q product accumulation. A branch of product accumulation derives from each of the two original single-stranded templates. Four types of products accumulate. Segments P^{C}U (235 bases) and Q^{C}D (221 bases) are the intermediate extension products which are not truncated and can be used as templates for further amplification. Segments P^{C}Q^{C} and Q^{C}P^{C} (each 170 bases) are terminal products which are truncated and cannot be amplified further by Q^{C} and P^{C}.

### TA-geometric (TA-g)

In one embodiment, TA-g amplifies a target nucleic acid contained in a sample, which method comprises (A) treating the sample under denaturing conditions to produce an upstream-directed original template and a downstream-directed original template from the target nucleic acid; (B) treating the upstream-directed original template with a truncating oligonucleotide as a primer, along with nucleoside triphosphates and a polymerase under suitable conditions, such that the primer hybridizes to a portion of the template and a downstream-directed, intermediate extension product of the primer is synthesized which is complementary to the template; (C) treating the downstream-directed original template with an oligonucleotide as a primer, along with nucleoside triphosphates and a polymerase under suitable conditions, such that the primer hybridizes to a portion of the template and an upstream-directed, intermediate extension product of the primer is synthesized which is complementary to the template; (D) treating the sample again under denaturing conditions to separate the primer extension products from the original templates; (E) treating the upstream-directed original template with a truncating oligonucleotide as a primer, along with nucleoside triphosphates and a polymerase under suitable conditions, such that the primer hybridizes to a portion of the template and a downstream-directed, intermediate extension product of the primer is synthesized which is complementary to the template; (F) treating the downstream-directed, intermediate extension product of (B) with an oligonucleotide as a primer, along with nucleoside triphosphates and a polymerase under suitable conditions, such that the primer hybridizes to a portion of the intermediate extension product and an upstream-directed extension product of the primer is synthesized which is complementary to the intermediate extension product of (B), wherein extension of the primer can be truncated, thereby resulting in a truncated extension product; (G) treating the downstream-directed original template with an oligonucleotide as a primer, along with nucleoside triphosphates and a polymerase under suitable conditions, such that the primer hybridizes to a portion of the template and an upstream-directed, intermediate extension product of the primer is synthesized which is complementary to the template; (H) treating the upstream-directed, intermediate extension product of (C) with a truncating oligonucleotide as a primer, along with nucleoside triphosphates and a polymerase under suitable conditions, such that the primer hybridizes to a portion of the extension product of (C) and a downstream-directed, intermediate extension product of the primer is synthesized which is complementary to the extension product of (C); (I) treating the sample again under denaturing conditions to separate the primer extension products from their templates; (J) thereafter treating during any subsequent primer extension reactions, each optionally followed by a denaturation and separation step, all upstream-directed original templates and upstream-directed, intermediate extension products with a truncating oligonucleotide as a primer to produce additional intermediate, non-truncated extension products of the primer which are complementary to the upstream-directed original templates and upstream-directed, intermediate extension products, respectively, and treating all downstream-directed original templates with an oligonucleotide as a primer to produce additional intermediate, non-truncated extension products of the primer which are complementary to the downstream-directed original template, and treating all downstream-directed, intermediate extension products with an oligonucleotide as a primer from which truncated extension products of the primer can be produced which are complementary to the downstream-directed, intermediate extension products, and wherein a sufficient number of amplification cycles occur to result in detectable amplification of the target nucleic acid.

The detectable amplification corresponds to a measure of the truncated extension products and/or intermediate extension products produced, as shown in the figures.

In another embodiment, the downstream-directed original template is instead treated with a truncating oligonucleotide as a primer, while the upstream-directed original template is treated with a regular oligonucleotide as a primer.

The first embodiment above is illustrated by the following preferred embodiment. However, the invention is not limited to the use of the chimeric oligonucleotide described below, but instead may be practiced with other types of truncating oligonucleotides, as defined herein.

In a preferred embodiment, TA-geometric (TA-g) utilizes one chimeric oligonucleotide and one regular oligonucleotide as primers. Four types of intermediate extension products and terminal products are produced in an asymmetric manner (FIG. 2A). The QP^{C} products are terminal after two or three cycles of replication from the original template. In the first cycle, P^{C} and Q are hybridized with UD and DU, respectively. They are extended to generate the intermediate extension products P^{C}U and QD (235 and 221 bases). In the second cycle, Q is hybridized with P^{C}U and is extended to generate a truncated extension product QP^{C} (170 bases); P^{C} is hybridized with QD and is extended to generate the intermediate extension product P^{C}Q (186 bases) (FIG. 1C). In the third cycle, Q is hybridized with P^{C}Q and is extended to generate the truncated extension product QP^{C} (170 bases). In this way, QP^{C} product can accumulate preferentially and DU strand is predominantly amplified (FIG. 3B).

FIG. 2A shows a schematic diagram of TA-geometric with one chimeric primer (P^{C}). TA-g is performed with P^{C} and Q. P^{C}U (235 bases), QD (221 bases) and P^{C}Q (186 bases) are the intermediate, non-truncated extension products. QP^{C} (170 bases) is the terminal product. By cycle 4, the maximal product accumulations are: P^{C}U=4, QD=4, PQ^{C}=6 and QP^{C}=10. By 40 cycles, P^{C}U or QD=40, P^{C}Q=780, and total QP^{C}=10699. Alternatively, TA-g can be performed with P and Q^{C} (not shown)

FIG. 2B shows a flowchart of TA-g product accumulation. A branch of product accumulation derives from each of the original single-stranded templates in an asymmetric manner. There are four types of products. Segments P^{C}U (235 bases), QD (221 bases) and P^{C}Q (186 bases) are the intermediate extension products. Segment QP^{C} (170 bases) is the terminal product.

FIGS. 3A and B contain graphs showing TA product accumulations of TA-q (A) and TA-g (B). On the X axis is the number of cycles. On the Y axis is the maximum number of each product molecules amplified from one single-stranded template by TA-q with P^{C} and Q^{C} or by TA-g with P^{C} and Q. Intermediate extension products and terminal products are indicated. In FIG. 3A, the amount of P^{C}U or Q^{C}D=C₀×λ×n, the amount of P^{C}Q^{C} or Q^{C}P^{C}=½×C₀×λ²×n×(n−1). The total amount of the four single-stranded products=C₀×[2×λ×n+λ²×n×(n−1)]. n is the total number of cycles. C₀ is the amount of the original single-stranded template (downstream=upstream). λ, the amplification efficiency, is the molecular ratio of the newly synthesized strand to the template strand (0<λ≦1). It is supposed that P^{C}, Q^{C}, P and Q have the same λ, and λ is constant with cycling. The total number of accumulated product molecules is the addition of all the types. With increasing cycles, P^{C}Q^{C} and Q^{C}P^{C} terminal products accumulate approximately proportional to n². In FIG. 3B, the amount of P^{C}U or QD=C₀×λ×n, the amount of P^{C}Q or QP^{C} from UD strand=½×C₀×λ²×n×(n−1), the amount of QP^{C} from DU strand=½×C₀×λ³×[(n−1) (n−2)+(n−2) (n−3)+(n−3) (n−4)+ . . . +2×1]. The total amount of the single-stranded products=C₀×{2×λ×n+λ²×n×(n−1)+½×λ³×[(n−1) (n−2)+(n−2) (n−3)+(n−3) (n−4)+ . . . +2×1]}. With increasing cycles, terminal product QP^{C} from DU strand accumulates approximately proportional to n³ and dominate

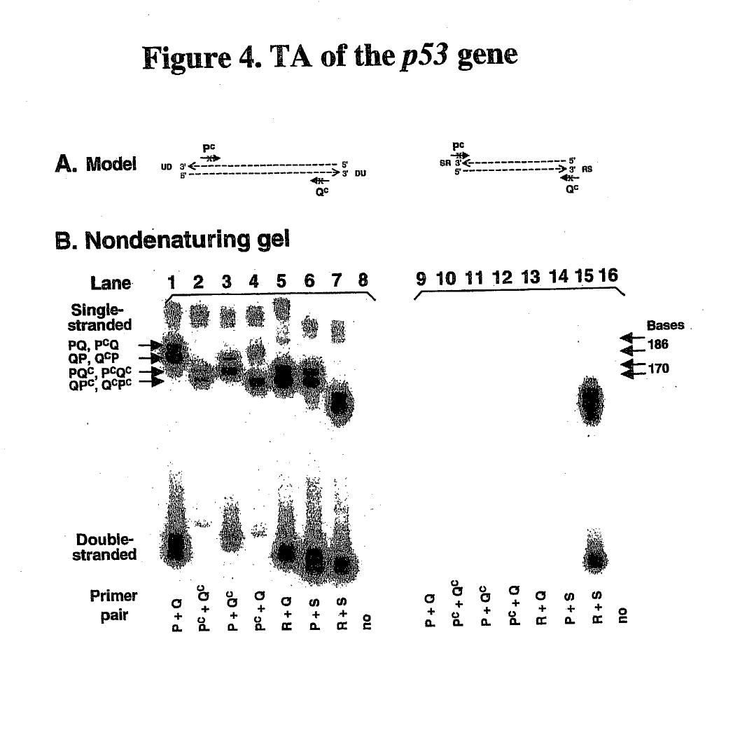

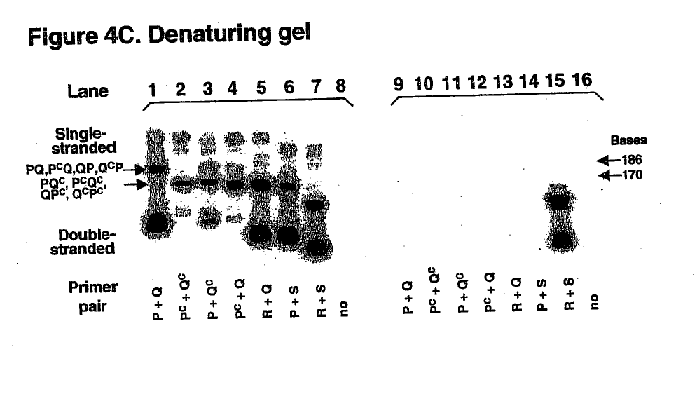

FIGS. 4A-C contain schematic diagrams and autoradiograms relating to TA of the p53 gene. In FIG. 4A, DU:UD (270 bp) is the duplexed template used in Lanes 1-8. RS:SR (154 bp) is the shortened duplexed template used in Lanes 9-16. RS and SR strands have only six 3′ nucleotides complementary to Q^{C} and P^{c}, respectively, which are the same as P^{C}Q^{C} or PQ^{C} and Q^{C}P^{C} or QP^{C}. The two above templates were amplified with seven sets of primer pairs (Table 1). After amplification, the products were electrophoresed through a non-denaturing and a denaturing gel. FIGS. 4B and 4C show autoradiograms of an 8% HR1000 nondenaturing SSCP gel and an 8% PAGE/7M urea denaturing gel, respectively. When the template DU:UD is used, Lane 2 to 4 are TA-q with P^{C} and Q^{C}, TA-g with P and Q^{C}, and TA-g with P^{C} and Q. Lanes 1, 5, 6 and 7 are control PCR's that generate size markers. When the shortened template RS:SR is used, no TA amplification occurs in Lanes 10, 11 and 12, demonstrating that the putative terminal products do not support amplification. Lanes 9, 13, 14 and 15 are PCR controls. Lane 8 and 16 are negative control without primers.

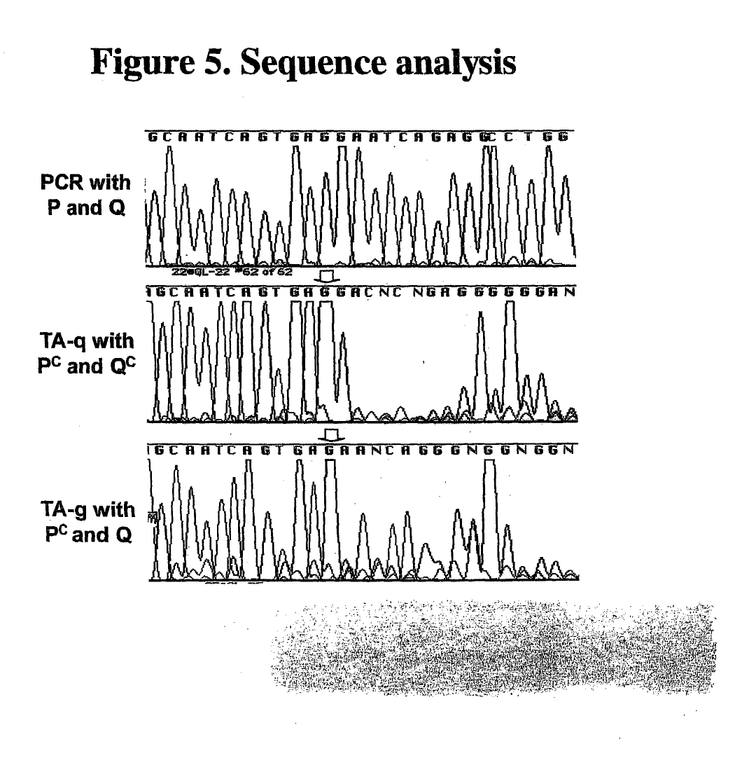

FIG. 5 shows a sequence analysis comparing PCR with TA-q and TA-g. The TA products amplified with P^{C} and Q^{C}, and P^{C} and Q were sequenced with the internal upstream primer in comparison with PCR product with P and Q. The truncation site is indicated by the arrow.

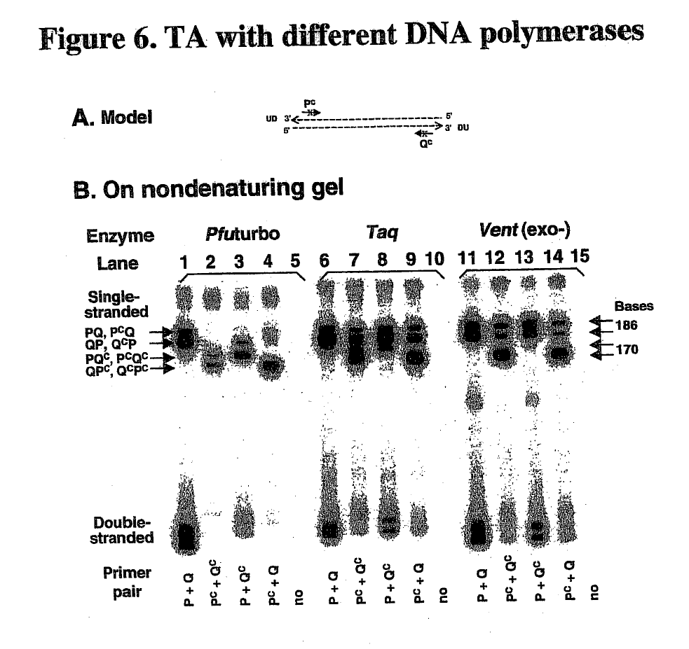

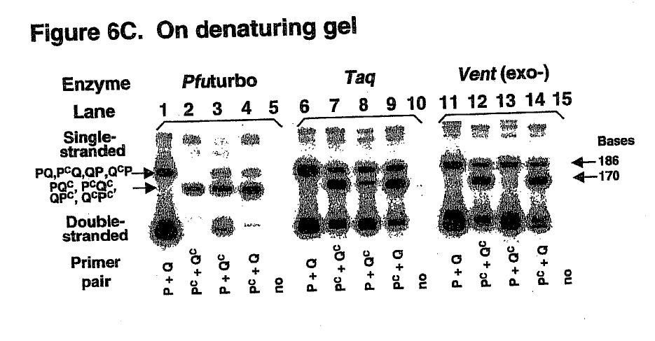

FIGS. 6A-C show schematic models and autoradiograms relating to TA with different DNA polymerases. In FIG. 6A, the DU:UD duplexed template (270 bp) is used. FIGS. 6B and 6C show autoradiograms of an 8% HR1000 nondenaturing SSCP gel and an 8% PAGE/7M urea denaturing gel. Pfuturbo, Taq and Vent(exo⁻) polymerases were used in Lanes 1-5, Lanes 6-10 and Lanes 11-15, respectively. Lanes 2, 7 and 12 are TA-q with P^{C} and Q^{C} Lanes 3, 8 and 13 are TA-g with P and Q^{C}. Lanes 4, 9 and 14 are TA-g with P^{C} and Q. Lanes 1, 6 and 11 are PCR controls. Lanes 5, 10 and 15 are negative controls with no primers.

TABLE 1

Oligonucleotides

| | | | | | |
|---|---|---|---|---|---|
| # | Name | Sequence 5-3′ | Chimera | Truncating | Concenrration (μM) |

| | | | | | |
|---|---|---|---|---|---|
| P | P53 (1) 22D^{a} | CCAGGCCTCTGATTCCTCACTG | No | No | 0.05 |
| Q | p53 (186) 22U | CTTAACCCCTCCTCCCAGAGAC | No | No | 0.1 |
| P^{C} | C*p53 (1) 22D | CCAGGCCTCTGAT [symbol] TCACTG^{b} | Yes | Yes | 2^{c} |
| Q^{C} | C*p53 (186) 22U | CTTAACCCCTCCT [symbol] AGAGAC^{b} | Yes | Yes | 0.05^{c} |
| R | p53 (16) 22D | TCACTGATTGCTCTTAGGTCTG | No | No | 0.1 |
| S | p53 (170) 22U | AGAGACCCCAGTTGCAAACCAG | No | No | 0.1 |

^{a}The sequence of the p53 gene was Genbank accession X54156 and nucleotide position 13287 is assigned as 1. As an example for oligonucleotide P, p53 = the p53 gene, (1) 22D = 5′ end of the primer begins at 1, and the length is 22 nucleotides “downstream” (d) (i.e., in the direction of transcription). The precise sizes and locations of the amplified fragment can be obtained from the informative names.
^{b}each truncating chimeric oligonucleotide of P^{C} and Q^{C} contain three 3′-5′ reversed-deoxynucleotides or 2′-OMe-ribonucleotides. They are located at 6, 7 and 8 nucleotides from the 3′ terminus (assigned as) and are indicated as bold, italic and underlined cases.
^{c}the concentration of 3′-5′ reversed-deoxynucleotide chimeric oligonucleotide. The concentrations of 2′-OME-ribonucleotide chimeric oligonucleotides P^{C} and Q^{C} are 0.1 μM and 2 μM.

The present invention also provides a kit for performing TA.

In one embodiment, the kit comprises one or more truncating oligonucleotides as primers, optionally an oligonucleotide as a primer, nucleoside triphosphates, a DNA polymerase, and suitable amplification reagents.

In a preferred embodiment, the one or more truncating oligonucleotides are chimeric oligonucleotides. The kit may also comprise a probe.

Applications of TA

TA has many possible applications. For example, when PCR amplifies from single cells, allele-dropout of one of the two chromosomal templates and mutation due to polymerase errors can confound the interpretation of the results (Persson, A. E. et al., 2000). To decrease the chances of allele drop out and polymerase error during single cell amplifications, TA-q can be performed to generate a reasonable number of amplified copies (e.g., 1,000). This can be followed by PCR to generate a desired amount of product.

With the recent advent of a practical high output in vitro translation system (Rapid Translation System 500, Roche), amplification of cDNA segments followed by in vitro transcription/translation can generate milligrams of proteins for structural and functional analysis, including protein crystallography (Biochemica No. 1, 20-23, 2001). During the development of protein drugs, it may be advantageous to determine amino acid sequences by analyzing the amino- or carboxy-terminus of the protein, but it is necessary for most of the amplified products to be completely error-free. TA should substantially increase the number of error-free products.

RNA amplification with in vitro translation (RAWIT), which was described as a PCR protocol (Sarkar, G. et al., 1989) can be adapted to TA. With the advent of highly parallel synthesis of oligonucleotides, RAWIT can generate the above-mentioned protein derivatives in a very rapid manner with the advantage that substantially more of the products for in vitro translation are error-free.

TA may also be adapted for PAP to generate TA-PAP, in which one or two oligonucleotide primers are truncating oligonucleotides that are pyrophosphorolysis activatable (Liu and Sommer, 2000).

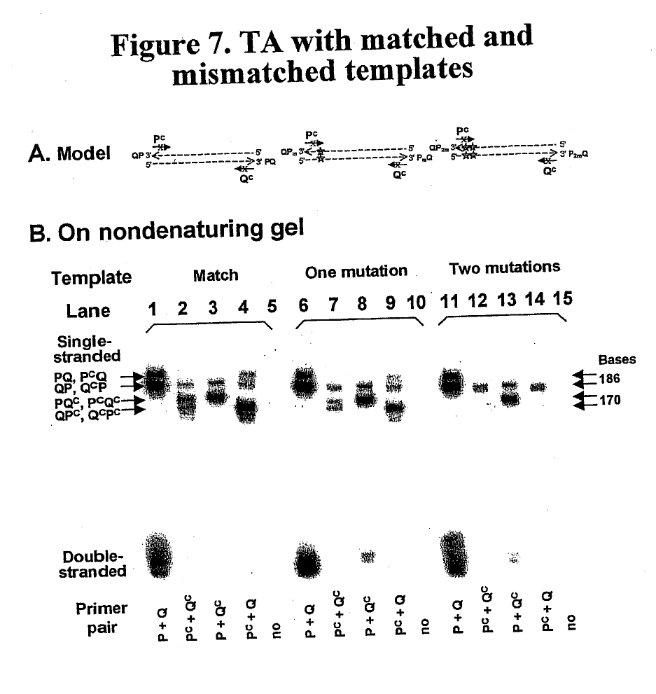

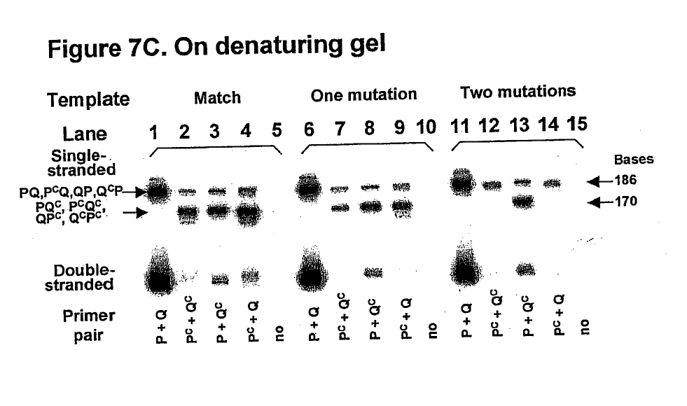

TA may also be adapted for allele-specific amplification to detect mutations. In one test, TA-q and TA-g were used for detection of known mutations (FIG. 7). Three types of duplexed templates PQ:QP, PₘQ:QPₘ and P₂ₘQ:QP₂ₘ (each 186 bp) were constructed to contain the wild type sequence, a G to T mutant at nucleotide position 22 and a double-mutant of G-T at T-A at nucleotide positions 17 and 22; so that P or P^{C} perfectly matched PQ:QP template, mismatched PₘQ:QPₘ template with the G to T mutation at the 3′ terminus, and mismatched with the two T to A and G to T mutations at −5 nucleotide from the 3′ terminus and at the 3′ terminus, respectively (FIG. 7A). The truncated products PQ^{C}, P^{C}Q^{C}, QP^{C} and Q^{C}P^{C} (each 170 bases) and the intermediate products PQ, P^{C}Q, QP and Q^{C}P (each 186 bases) are shown in FIGS. 7B and C. Compared with a perfect match, TA-q was inhibited by one mismatch at the 3′ terminus and was severely inhibited by the two mismatches, showing the specificity for mutation detection. For TA-g with P and Q^{C}, the amplification was not inhibited by one mismatch or two mismatches. For another TA-g with P^{C} and Q, the amplification was inhibited by one mismatch and was severely inhibited by two mismatches, showing the specificity. One TA-g with P^{C} and Q shows an asymmetric inhibition.

FIG. 7A shows a model of the p53 gene, which was examined using Pfuturbo polymerase. The wild type template PQ:QP, a mutant template PₘQ:QPₘ and a double-mutant template P₂ₘQ:QP₂ₘ are indicated (each duplex 186 bp). The * means a mutation on the template. FIGS. 7B and 7C show autoradiograms of an 8% HR1000 SSCP nondenaturing gel and an 8% PAGE/7M urea denaturing gel. P or P^{C} perfectly matched the wild type template PQ:QP in Lanes 1-5; P or P^{C} mismatched the mutant template PₘQ:QPₘ with the G to T mutation at the 3′ terminus in Lanes 6-10; P or P^{C} mismatched the double-mutant template P₂ₘQ:QP₂ₘ with the two T to A and G to T mutations at −5 nucleotide from the 3′ terminus and at the 3′ terminus in Lanes 11-15. TA-q with the P^{C} and Q^{C} is in Lanes 2, 7 and 12. TA-g with P and Q^{C} is in Lanes 3, 8 and 13. Another TA-g with P^{C} and Q is in Lanes 4, 9 and 14. PCR controls are in Lanes 1, 6 and 11. Negative controls with no primers are in Lanes 5, 10 and 15.

The TA products were further analyzed by an ABI fluorescence sequencer and the results showed that the mismatched G at the 3′ terminus of the primer was removed by the 3′-5′ exonuclease and a complementary T nucleotide is incorporated by the polymerase, but the mismatched A at −5 nucleotide from the 3′ terminus of the primer was not removed (data not shown)

TA vs. PCR

The exponential nature of PCR amplification can generate a great deal of product. However, this can be problematic for quantitative PCR, because small variations in efficiency per cycle can have dramatic effects on the yields after a typical 30 cycles (Liu, Q. et al., 1997). This limitation is substantial in clinical practice since large heterozygous deletions in patients are commonly missed because only two fold differences in template concentrations are difficult to detect. The quadratic and geometric nature of TA, although useful qualitatively, is better suited for quantitative measurements. For example, TA-g can be used for preferential amplification of only one specific strand of the duplexed template to analyze strand-specific events, such as adduct or DNA damage (Pfeifer, G. P., et al., 1999), and to perform Sanger sequencing (Sanger, F. et al., 1977) or sequencing by hybridization (Lysov, I. et al.; Southern, E. M. et al., 1992; Hacia, J. G. et al., 1999). TA also can be combined with bidirectional denaturing fingerprinting (Liu, Q. et al., 1998).

TA also offers advantages over PCR. For example, there are many applications for which PCR is the method of choice because of convenience of the existing infrastructure of established protocols, and most importantly, the greater than one million fold amplification readily achieved. However, in commercial situations, the application of PCR may be prohibited or not optimal due to licensing or royalty considerations. Hence, there is great interest in alternative forms of amplification (Baner, J. et al., 1998; Barany, F., 1991; Guatelli, J. C., 1990; Kramer, F. R., 1989; Landegren, U. et al., 1988; Liu, Q. et al., 2000; Lizardi, P. M., et al., 1998). TA is such an alternative method that retains two important aspects of PCR: the flexibility of amplification of any sequence and the convenience of automation by thermal cycling.

In the broadest sense, PCR is an exponential process in which the products generated in each cycle are templates for amplification in subsequent cycles (Mullis, K. B., 1987). TA retains certain features of PCR, such as the use of a pair of opposite primers and automation by thermal cyclers. But TA differs from PCR in important ways: i) TA uses truncating oligonucleotides; ii) TA produces truncated terminal products that generally do not function as templates for further amplification, iii) TA amplifies quadratically (TA-q) or geometrically (TA-g); iv) the products in most, if not all, cases are never more than three rounds of replication from the original template, providing more error-free products by dramatically limiting the propagation of polymerase errors; v) in TA-g, primers of equal concentration produce an asymmetric amplification. The excess of single-stranded product produced by TA-g may be advantageous for probes in other analyses. In asymmetric PCR the amount of excess single-stranded product is hard to control because it varies dramatically with template and primer concentrations. TA-g is a more robust method of asymmetric amplification than asymmetric PCR in which a single-stranded product may be amplified through both an exponential and then linear stage wherein one primer has been exhausted using unequal amounts of primers (Gyllensten, U. B. et al., 1988).

The present invention is further illustrated by the following examples, which are not intended to be limiting.

### EXAMPLE 1

### Preparation of Templates by PCR

Each segment of exon 6 in the human p53 gene (Genbank accession X54156 and nucleotide 13287 is assigned as position 1) was amplified by PCR. A 270 bp duplexed segment (DU:UD) of 57.4% GC content was prepared with primers of D=p53(−35)30D (5′GGT GAG CAG CTG GGG CTG GAG AGA CGA CAG3′) and U=p53(235)30U (5′CCT ACT GCT CAC CCG GAG GGC CAC TGA CAA3′); A 154 bp segment (RS:SR) was prepared with primers of R=p53(16)22D and S=p53(170)22U (Table 1). The volume of the PCR reaction is 50 μl: 50 mM KCl, 10 mM Tris/HCl, pH 8.3, 1.5 mM MgCl₂, 200 μM each of the four dNTPs, 0.1 μM of each primer, 2% DMSO, 1 U of Taq DNA polymerase (Roche) and 250 ng of genomic DNA. Cycling conditions were 94° C. for 15 sec., 55° C. for 30 sec., and 72° C. for one min., for a total of 35 cycles with a GeneAmp PCR System 9700 (Perkin Elmer Applied Biosystems). The PCR product was purified from primers and other small molecules by approximately 10,000-fold by three rounds of retention on a Centricon® 100 microconcentrator (Amicon). The amount of recovered PCR product was determined by UV absorbance at 260 nm.

### EXAMPLE 2

### Synthesis of Chimeric Oligonucleotides

Synthesis of chimeric oligonucleotides (Table 1) was performed on a model 394 DNA/RNA Synthesizer (Applied Biosystems) in a DMT-on mode (Gait, M. J., 1984). Extended time (500 sec.) was used for the coupling of 2′-OMe (C and T)- 3′-CE-phosphoramidites and 300 sec. for dCBZ and T 5′-CE-phosphoramidites (Glen Research). The T, dCTAC, dGTAC, dATAC 3′-CE-phosphoroamidites and an activator (0.25 M of 4,5-dicyanoimidazole in acetonitrile) were from Proligo. The product was cleaved from the resin and deprotected. DMT-on products were separated on preparative PRP-1 (Hamilton) column. After the removal of DMT, the chimeras were purified by Ion-Paired HPLC (Swiderski, P. M. et al., 1994). The amount of product was determined by UV absorbance at 260 nm. Purity of the product was confirmed using ³²P radioactive labeling by T4 kinase and 7M urea/analytical PAGE in TBE buffer (90 mM Tri/borate, 1 mM EDTA, pH 8.3).

### EXAMPLE 3

### Truncated Amplification

The DU:UD (270 bp) duplexed template prepared in Example 1 was amplified using two oligonucleotide primers, wherein at least one of the primers was a chimeric oligonucleotide. The reaction mixture contained a total volume of 25 μl: 20 mM Tris/HCl (pH 8.8), 10 mM KCl, 10 mM (NH₄)₂SO₄, 2.0 mM MgSO₄, 250 μM each of the four dNTPs (dATP, dTTP, dGTP and dCTP), downstream and upstream oligonucleotide primers (see Table 1 for final concentrations), 0.1% Triton®X-100, 0.1 mg/ml nuclease-free bovine serum albumin (BSA), 2% DMSO, 1 μCi of [α-³²P]-dCTP (3000 Ci/mmole, Amersham), 1 U of Pfuturbo DNA polymerase (Stratagene), and 2 ng of the template. The components were different with 1 U Taq polymerase: 50 mM KCl, 10 mM Tris/HCl (pH 8.3), 1.5 mM MgCl₂, 200 μM each of dNTPs and no BSA; or with 1 U Vent_{R}® (exo⁻) polymerase (BioLabs): no BSA. The cycling conditions were 94° C. for 20 sec., 55° C. for 30 sec., and 68° C. for 2 min. for a total of 20 cycles.

The product was electrophoresed through an 8% polyacrylamine/7M urea denaturing gel with 90 mM TBE buffer or through an 8% HR-1000™ (Genomyx) nondenaturing SSCP gel with 30 mM TRI/TRI buffer (Liu, Q. et al., 1999). The gel was dried and autoradiography was performed with Kodak X-OMAT™ AR film.

### EXAMPLE 4

### Sequence Analysis

The DU:UD template was amplified by TA using Pfuturbo DNA polymerase for 25 cycles and then purified by a Microcon® 50 microconcentrator (Amicon). Standard sequence analysis was performed using ABI 377 fluorescence sequencer and BigDye terminator chemistry with AmpliTaq FS DNA polymerase (PE Applied Biosystem). The two internal sequencing primers were p53(35)30D (5′CTG GCC CCT CCT CAG CAT CTT ATC CGA GTG3′) and p53(177)30U (5′TCC TCC CAG AGA CCC CAG TTG CAA ACC AGA3′). The data were analyzed by a Sequencher™ software (Gene Codes).

### EXAMPLE 5

### TA of the p53 Gene

The DU:UD (270 bp) duplexed template of exon 6 of the p53 gene was amplified using Pfuturbo polymerase (FIG. 4). For TA-q performed with P^{C} and Q^{C}, the truncated extension products P^{C}Q^{C} and Q^{C}P^{C} (each 170 bases) were generated. There is no product of 186 bases indicating Pfuturbo polymerase could not pass through the reversed-deoxyribonucleotides on the templated chimeras P^{C} and Q^{C}. For TA-g with P and Q^{C}, the intermediate extension product Q^{C}P (186 bases) and the truncated extension product PQ^{C} (170 bases) were generated. For another possible TA-g with P^{C} and Q, the intermediate ex-tension product P^{C}Q (186 bases) and the truncated extension product QP^{C} (170 bases) were generated.

A shortened template, RS:SR (154 bp), was designed to test that the truncated extension product is terminal (FIG. 4). RS and SR strands have only six 3′ nucleotides complementary to Q^{C} and P^{C}, respectively. They have the same nucleotide sequences as P^{C}Q^{C} or PQ^{C} and as Q^{C}P^{C} or QP^{C}. No products were produced by TA, indicating that the truncated product is the terminal product. Two other shortened templates, PS:SP (170 bp) and RQ:QR (170 bp), were also tested. PS and QR strands contain the same nucleotide sequences as P^{C}Q and Q^{C}P, respectively. Again, no products were amplified by TA from any of the shortened templates (data not shown).

P^{C}Q and P^{C}Q^{C} can be separated from QP^{C} and Q^{C}P^{C} (each 170 bases), and P^{C}Q can be separated from Q^{C}P (each 186 bases) on a nondenaturing SSCP gel (FIG. 4B). The sense and antisense strands of the same size could not be separated on a denaturing gel (FIG. 4C). In the experiments for FIGS. 4B and C, the double-stranded products were also seen. They are due to hybridization among the single-stranded molecules in the electrophoresis, because the double-stranded products did not appear on a more powerful denaturing gel containing 40% formamide and 7M urea (data not shown).

The TA products were further analyzed by ABI fluorescent sequence analysis with internal primers in the downstream and upstream directions. The sequence result shows the correct sequences and truncated sites (FIG. 5), confirming that the TA products were as thought and demonstrating that TaqFS DNA polymerase does extend through the modified nucleotides at a low rate.

### EXAMPLE 6

### Effect of DNA Polymerase

The DU:UD (270 bp) template was amplified by Pfuturbo, Taq and Vent (exo⁻) polymerases. Taq and Vent (exo⁻) can partially read through the analogue nucleotides of the templated chimeras, but not Pfuturbo (FIG. 6). For TA-q with P^{C} and Q^{C} the truncated extension products P^{C}Q^{C} and Q^{C}P^{C} (each 170 bases) were generated. However, the full length products (186 bases) were also generated by reading through the analogous nucleotides by Taq and Vent(exo⁻) polymerases. For TA-g with P and Q^{C}, the intermediate extension product Q^{C}P (186 bases) and the truncated extension product PQ^{C} (170 bases) were generated. However, the full length products were also generated. For another TA-g with P^{C} and Q, the intermediate extension product P^{C}Q (186 bases) and the truncated extension product QP^{C} (170 bases) were generated, but the full length products were also generated.

### EXAMPLE 7

### Chimeras Containing 2′-OMe-ribonucleotides

The above experiments were performed with other truncating chimeric oligonucleotides which contain three successive 2′-OMe-ribonucleotides at 6, 7 and 8 nucleotides from the 3′ terminus (Table 1). The results were similar in that Pfuturbo polymerase could not read through the modified nucleotides on the template, but Taq, Vent (exo⁻), and AmpliTaqFS could partially extend through the modified nucleotides (data not shown). It was reported that some DNA polymerases, such as Taq, have reverse transcription activity and can use a RNA template for extension (Jones, et al., 1989; Shibata et al., 1995).

### EXAMPLE 8

### Preparation of Templates by PCR

A 186 bp segment (PQ:QP) of 52.7% GC content was prepared by PCR, using the same conditions as in Example 1, with primers of P=p53(1)22D and Q=p53(186)22U; Another 186 bp segment (PₘP:QPₘ) which contains a G-T mutation at position 22 was prepared with primers of Pₘ=p53(1)30Dₘ (5′CCA GGC CTC TGA TTC CTC ACT TAT TGC TCT3′,T is a designed mutation at 8 nucleotides from the 3′ terminus) and Q=p53(186)22U; A third 186 bp segment (P₂ₘQ:QP₂ₘ) which contains two mutations of G-T at T-A at positions 17 and 22 was prepared with primer pair of p53(1)3OD₂ₘ(5′CCA CGC CTC TGA TTC CAC ACT TAT TGC TCT3′, T and A are two designed mutations at 8 and 13 nucleotides from the 3′ terminus) and Q=p53(186)22U.

The publications and other materials cited herein to illuminate the background of the invention and to provide additional details respecting the practice of the invention are incorporated herein by reference to the same extent as if they were individually indicated to be incorporated by reference.

While the invention has been disclosed by reference to the details of preferred embodiments of the invention, it is to be understood that the disclosure is intended in an illustrative rather than a limiting sense, as it is contemplated that modifications will readily occur to those skilled in the art, within the spirit of the invention and the scope of the appended claims.

References

1. Baner, J., M. Nilsson, M. Mendel-Hartvig and U. Landegren. 1998. Signal amplification of padlock probes by rolling circle replication. Nucleic Acids Res. 26: 5073-5078.

2. Barany, F. 1991. Genetic disease detection and DNA amplification using cloned thermostable ligase. Proc. Natl. Acad. Sci. USA. 88: 189-193.

3. Gait, M. J. 1984. Oligonucleotide synthesis: a practical approach. IRL Press, Oxford, Washington, D.C.

4. Guatelli, J. C., K. M. Whitfield, D. Y. Kwoh, K. J. Barringer, D. D. Richman and T. R. Gingeras. 1990. Isothermal, in vitro amplification of nucleic acids by a multienzyme reaction modeled after retroviral replication. Proc. Natl. Acad. Sci. USA. 87: 1874-1878.

5. Gyllensten, U. B. and H. A. Erlich. 1988. Generation of single-stranded DNA by the polymerase chain reaction and its application to direct sequencing of the HLA-DQA locus. Proc. Natl. Acad. Sci. USA. 85: 7652-7656.

6. Hacia, J. G. and F. S. Collins. 1999. Mutational analysis using oligonucleotide microarrays. J. Med. Genet. 36: 730-736.

7. Jones, M. D. and N. S. Foulkes. 1989 Reverse transcription of mRNA by Thermus aquaticus DNA polymerase. Nucleic Acids Res. 17: 8387-8388.

8. Kramer, F. R. and P. M. Lizardi. 1989. Replicatable RNA reporters. Nature. 339: 401-402.

9. Landegren, U., R. Kaiser, J. Sanders and L. Hood. 1988. A ligase-mediated gene detection technique. Science. 241: 1077-1080.

10. Liu, Q., X. Li and S. S. Sommer. 1999. pk-matched running buffers for gel electrophoresis. Anal. Biochem. 270: 112-122.

11. Liu, Q. and S. S. Sommer. 2000. Pyrophosphorolysis activated polymerization (PAP): application to allele-specific amplification. BioTechniques. 29: 1072-1080.

12. Liu, Q., E. C. Thorland and S. S. Sommer. 1997. Inhibition of PCR amplification by a point mutation downstream of a primer. BioTechniques. 22: 292-298.

13. Liu, Q., B. G. Weinshenker, D. M. Wingerchuk and S. S. Sommer. 1998. Denaturation fingerprinting: two related mutation detection methods especially advantageous for high G+C regions. BioTechniques. 24: 140-147.

14. Lizardi, P. M., X. Huang, Z. Zhu, P. Bray-Ward, D. C. Thomas and D. C. Ward. 1998. Mutation detection and single-molecule counting using isothermal rolling-circle amplification. Nat. Genet. 19: 225-232.

15. Lysov, I., V. L. Florent'ev, A. A. Khorlin, K. R. Khrapko and V. V. Shik. 1988. Determination of the nucleotide sequence of DNA using hybridization with oligonucleotides. A new method. Dokl Akad Nauk SSSR. 303: 1508-1511.

16. Misra, H. S., P. K. Pandey, M. J. Modak, R. Vinayak and V. N. Pandey. 1998. Polyamide nucleic acid-DNA chimera lacking the phosphate backbone are novel primers for polymerase reaction catalyzed by DNA polymerases. Biochemistry. 37: 1917-1925.

17. Mullis, K. B. 1987. Process for amplifying nucleic acid sequences. U.S. Pat. No. 4,683,202.

18. Newton, C. R., Graham, A., Heptinstall, L. E., Powell, S. J., Summers, C., Kalsheker, N., Smith, J. C. and Markham, A. F. (1989) Analysis of any point mutation in DNA. The amplification refractory mutation system (ARMS). Nucleic Acids Res., 17, 2503-2516.

19. Nichols, W. C. Liepnieks, J. J., McKusick, V. A. and Benson, M. D. (1989) Direct sequencing of the gene for Maryland/German familial amyloidotic polyneuropathy type II and genotyping by allele-specific enzymatic amplification. Genomics, 5, 535-540.

20. Persson, A. E., G. Ling, C. Williams, H. Backvall, J. Ponten, F. Ponten and J. Lundeberg. 2000. Analysis of p53 mutations in single cells obtained from histological tissue sections. Anal. Biochem. 287: 25-31.

21. Pfeifer, G. P., H. H. Chen, J. Komura and A. D. Riggs. 1999. Chromatin structure analysis by ligation-mediated and terminal transferase-mediated polymerase chain reaction. Methods Enzymol. 304: 548-571.

22. Saiki, R. K., S. Scharf, F. Faloona, K. B. Mullis, G. T. Horn, H. A. Erlich and N. Arnheim. 1985. Enzymatic amplification of beta-globin genomic sequences and restriction site analysis for diagnosis of sickle cell anemia. Science. 230: 1350-1354.

23. Sanger, F., S. Nichlen and A. R. Coulson. 1977. DNA sequencing with chain-terminating inhibitor. Proc. Natl. Acad. Sci. USA. 75: 5463-5467.

24. Sarkar, G. and S. S. Sommer. 1989. Access to a messenger RNA sequence or its protein product is not limited by tissue or species specificity. Science. 244: 331-334.

25. Shibata, H., T. Tahira, and K. Hayashi. 1995. RNA-primed PCR. Genome Res. 5: 400-4003.

26. Sommer, S. S., Cassady, J. D., Sobell, J. L. and Bottema, C. D. K. (1989) A novel method for detecting point mutations or polymorphisms and its application to population screening for carriers of phenylketonuria. Mayo Clinic Proc., 64, 1361-1372.

27. Southern, E. M., U. Maskos and J. K. Elder. 1992. Analyzing and comparing nucleic acid sequences by hybridization to arrays of oligonucleotides: evaluation using experimental models. Genomics. 13: 1008-1017.

28. Swiderski, P. M., E. L. Bertrand and B. E. Kaplan. 1994. Polystyrene reverse-phase ion-pair chromatography of chimeric ribozymes. Anal Biochem. 216: 83-88.

29. Wu, D. Y., Ugozzoli, L., Pal, B. K. and Wallace, R. B. (1989) Allele-specific enzymatic amplification of b-globin genomic DNA for diagnosis of sickle cell anemia. Proc. Natl. Acad. Sci. USA, 86, 2757-2760.

30. 2001. RTS 500-maximize the possibilities in protein expression. Biochemica. No.1, 20-23.

What is claimed is:

1. A method for amplifying a target nucleic acid contained in a sample, the method comprising:
(A) treating the sample under denaturing conditions to produce original single strand templates from the target nucleic acid;
(B) treating the original templates with a truncating oligonucleotide as a primer, along with nucleoside triphosphates and a polymerase under suitable conditions, such that the primers hybridize to a portion of the original templates and an extension product of the primers is synthesized which is complementary to the original templates;
(C) treating the sample again under denaturing conditions to separate the extension products from the original templates;
(D) treating the extension products and the original templates with a truncating oligonucleotide as a primer, along with nucleoside triphosphates and a polymerase under suitable conditions, such that the primers hybridize to a portion of the extension products and the original templates and an extension product of the primers is synthesized which is complementary to the extension products and the original templates, wherein extension of the primer on the extension products can be truncated, thereby producing a truncated extension product;
(E) treating the sample again under denaturing conditions to separate the extension products from their templates; and
(F) repeating steps (D) and (E) a sufficient number of times to result in detectable amplification of the target nucleic acid.

2. The method of claim 1, wherein the truncating oligonucleotide is a chimeric oligonucleotide.

3. The method of claim 1, wherein the target nucleic acid is DNA.

4. The method of claim 1, wherein the polymerase is a DNA polymerase.

5. The method of claim 4, wherein the DNA polymerase is Pfuturbo.

6. The method of claim 2, wherein the chimeric oligonucleotide comprises one or more 3′-5′ reversed-deoxynucleotides.

7. The method of claim 6, wherein the chimeric oligonucleotide comprises three 3′-5′ reversed-deoxynucleotides.

8. The method of claim 7, wherein the 3′-5′ reversed-deoxynucleotides are located about 6 to 8 nucleotides from the 3′ terminus of the oligonucleotide.

9. The method of claim 2, wherein the chimeric oligonucleotide comprises one or more 2′-OMe-ribonucleotides.

10. The method of claim 9, wherein the chimeric oligonucleotide comprises three 2′-OMe-ribonucleotides.

11. The method of claim 10, wherein the 2′-OMe-ribonucleotides are located about 6 to 8 nucleotides from the 3′ terminus of the oligonucleotide.

12. A method for amplifying a target nucleic acid contained in a sample, the method comprising:
(A) treating the sample under denaturing conditions to produce an upstream-directed original template and a downstream-directed original template from the target nucleic acid;
(B) treating the upstream-directed original template with a truncating oligonucleotide as a primer, along with nucleoside triphosphates and a polymerase under suitable conditions, such that the primer hybridizes to a portion of the template and a downstream-directed extension product of the primer is synthesized which is complementary to the template;
(C) treating the downstream-directed original template with an oligonucleotide as a primer, along with nucleoside triphosphates and a polymerase under suitable conditions, such that the primer hybridizes to a portion of the template and an upstream-directed extension product of the primer is synthesized which is complementary to the template;
(D) treating the sample again under denaturing conditions to separate the extension products from the original templates;
(E) treating the upstream-directed original template with a truncating oligonucleotide as a primer, along with nucleoside triphosphates and a polymerase under suitable conditions, such that the primer hybridizes to a portion of the template and a downstream-directed extension product of the primer is synthesized which is complementary to the template;
(F) treating the downstream-directed extension product of (B) with an oligonucleotide as a primer, along with nucleoside triphosphates and a polymerase under suitable conditions, such that the primer hybridizes to a portion of the extension product and an upstream-directed extension product of the primer is synthesized which is complementary to the extension product of (B), wherein extension of the primer can be truncated, thereby producing a truncated extension product;
(G) treating the downstream-directed original template with an oligonucleotide as a primer, along with nucleoside triphosphates and a polymerase under suitable conditions, such that the primer hybridizes to a portion of the template and an upstream-directed extension product of the primer is synthesized which is complementary to the template;
(H) treating the upstream-directed extension product of (C) with a truncating oligonucleotide as a primer, along with nucleoside triphosphates and a polymerase under suitable conditions, such that the primer hybridizes to a portion of the extension product of (C) and a downstream-directed extension product of the primer is synthesized which is complementary to the extension product of (C);
(I) treating the sample again under denaturing conditions to separate the extension products from their templates;
(J) thereafter treating during any subsequent primer extension reactions, each optionally followed by a denaturation and separation step, all upstream-directed original templates and upstream-directed extension products with a truncating oligonucleotide as a primer to produce non-truncated extension products of the primer which are complementary to the upstream-directed original templates and upstream-directed extension products, and treating all downstream-directed original templates with an oligonucleotide as a primer to produce non-truncated extension products of the primer which are complementary to the downstream-directed original template, and treating all downstream-directed extension products with an oligonucleotide as a primer from which truncated extension products of the primer can be produced which are complementary to the downstream-directed extension products, and wherein a sufficient number of amplification cycles occur to result in detectable amplification of the target nucleic acid.

13. The method of claim 12, wherein the truncating oligonucleotide is a chimeric oligonucleotide.

14. The method of claim 12, wherein the target nucleic acid is DNA.

15. The method of claim 12, wherein the polymerase is a DNA polymerase.

16. The method of claim 15, wherein the DNA polymerase is Pfuturbo.

17. The method of claim 13, wherein the chimeric oligonucleotide comprises one or more 3′-5′ reversed-deoxynucleotides.

18. The method of claim 17, wherein the chimeric oligonucleotide comprises three 3′-5′ reversed-deoxynucleotides.

19. The method of claim 18, wherein the 3′-5′ reversed-deoxynucleotides are located about 6 to 8 nucleotides from the 3′ terminus of the oligonucleotide.

20. The method of claim 13, wherein the chimeric oligonucleotide comprises one or more 2′-OMe-ribonucleotides.

21. The method of claim 20, wherein the chimeric oligonucleotide comprises three 2′-OMe-ribonucleotides.

22. The method of claim 21, wherein the 2′-OMe-ribonucleotides are located about 6 to 8 nucleotides from the 31 terminus of the oligonucleotide.

23. A method for amplifying a target nucleic acid contained in a sample, the method comprising:
(A) treating the sample under denaturing conditions to produce an upstream-directed original template and a downstream-directed original template from the target nucleic acid;
(B) treating the downstream-directed original template with a truncating oligonucleotide as a primer, along with nucleoside triphosphates and a polymerase under suitable conditions, such that the primer hybridizes to a portion of the template and an upstream-directed extension product of the primer is synthesized which is complementary to the template;
(C) treating the upstream-directed original template with an oligonucleotide as a primer, along with nucleoside triphosphates and a polymerase under suitable conditions, such that the primer hybridizes to a portion of the template and a downstream-directed extension product of the primer is synthesized which is complementary to the template;
(D) treating the sample again under denaturing conditions to separate the extension products from the original templates;
(E) treating the downstream-directed original template with a truncating oligonucleotide as a primer, along with nucleoside triphosphates and a polymerase under suitable conditions, such that the primer hybridizes to a portion of the template and an upstream-directed extension product of the primer is synthesized which is complementary to the template;
(F) treating the upstream-directed extension product of (B) with an oligonucleotide as a primer, along with nucleoside triphosphates and a polymerase under suitable conditions, such that the primer hybridizes to a portion of the extension product and a downstream-directed extension product of the primer is synthesized which is complementary to the extension product of (B), wherein extension of the primer can be truncated, thereby producing a truncated extension product;
(G) treating the upstream-directed original template with an oligonucleotide as a primer, along with nucleoside triphosphates and a polymerase under suitable conditions, such that the primer hybridizes to a portion of the template and a downstream-directed extension product of the primer is synthesized which is complementary to the template;
(H) treating the downstream-directed extension product of (C) with a truncating oligonucleotide as a primer, along with nucleoside triphosphates and a polymerase under suitable conditions, such that the primer hybridizes to a portion of the extension product of (C) and an upstream-directed extension product of the primer is synthesized which is complementary to the extension product of (C);
(I) treating the sample again under denaturing conditions to separate the extension products from their templates;
(J) thereafter treating during any subsequent primer extension reactions, each optionally followed by a denaturation and separation step, all downstream-directed original templates and downstream-directed extension products with a truncating oligonucleotide as a primer to produce non-truncated extension products of the primer which are complementary to the downstream-directed original templates and downstream-directed extension products, and treating all upstream-directed original templates with an oligonucleotide as a primer to produce non-truncated extension products of the primer which are complementary to the upstream-directed original template, and treating all upstream-directed extension products with an oligonucleotide as a primer from which truncated extension products of the primer can be produced which are complementary to the upstream-directed extension products, and wherein a sufficient number of amplification cycles occur to result in detectable amplification of the target nucleic acid.

24. The method of claim 23, wherein the truncating oligonucleotide is a chimeric oligonucleotide.

25. The method of claim 23, wherein the target nucleic acid is DNA.

26. The method of claim 23, wherein the polymerase is a DNA polymerase.

27. The method of claim 26, wherein the DNA polymerase is Pfuturbo.

28. The method of claim 24, wherein the chimeric oligonucleotide comprises one or more 3′-5′ reversed-deoxynucleotides.

29. The method of claim 28, wherein the chimeric oligonucleotide comprises three 3′-5′ reversed-deoxynucleotides.

30. The method of claim 29, wherein the 3′-5′ reversed-deoxynucleotides are located about 6 to 8 nucleotides from the 3′ terminus of the oligonucleotide.

31. The method of claim 24, wherein the chimeric oligonucleotide comprises one or more 2′-OMe-ribonucleotides.

32. The method of claim 31, wherein the chimeric oligonucleotide comprises three 2′-OMe-ribonucleotides.

33. The method of claim 32, wherein the 2′-OMe-ribonucleotides are located about 6 to 8 nucleotides from the 3′ terminus of the oligonucleotide.

34. A method for amplifying a target nucleic acid contained in a sample, the method comprising:
(A) treating the nucleic acid with a truncating oligonucleotide as a primer, along with nucleoside triphosphates and a polymerase under suitable conditions, such that the primer hybridizes to the nucleic acid and an extension product of the primer is synthesized which is complementary to the nucleic acid;
(B) treating the sample under denaturing conditions to separate the extension product from the original template;
(C) treating the original template with a truncating oligonucleotide as a primer, along with nucleoside triphosphates and a polymerase under suitable conditions, such that the primer hybridizes thereto and an extension product of the primer is synthesized which is complementary with the original template;
(D) treating the extension product with an oligonucleotide or a truncating oligonucleotide as a primer, along with nucleoside triphosphates and a polymerase under suitable conditions, such that the primer hybridizes thereto and an extension product of the primer is synthesized which is complementary to the extension product, wherein extension of the primer can be truncated, thereby producing a truncated extension product;
(E) treating the sample again under denaturing conditions to separate each of the extension products from its template; and
(F) repeating steps (C) to (E) a sufficient number of times to result in detectable amplification of the target nucleic acid.

35. The method of claim 34, wherein the truncating oligonucleotide is a chimeric oligonucleotide.

36. The method of claim 34, wherein said target nucleic acid is double-stranded and its strands are separated by denaturation before or during step (A).

37. The method of claim 34, wherein said target nucleic is single-stranded.

38. The method of claim 34, wherein the target nucleic acid is DNA.

39. The method of claim 34, wherein the polymerase is a DNA polymerase.

40. The method of claim 39, wherein the DNA polymerase is Pfuturbo.

41. The method of claim 35, wherein the chimeric oligonucleotide comprises one or more 3′-5′ reversed-deoxynucleotides.

42. The method of claim 41, wherein the chimeric oligonucleotide comprises three 3′-5′ reversed-deoxynucleotides.

43. The method of claim 42, wherein the 3′-5′ reversed-deoxynucleotides are located about 6 to 8 nucleotides from the 3′ terminus of the oligonucleotide.

44. The method of claim 35, wherein the chimeric oligonucleotide comprises one or more 2′-OMe-ribonucleotides.

45. The method of claim 44, wherein the chimeric oligonucleotide comprises three 2′-OMe-ribonucleotides.

46. The method of claim 45, wherein the 2′-OMe-ribonucleotides are located about 6 to 8 nucleotides from the 3′ terminus of the oligonucleotide.

47. A method for producing a truncated nucleic acid during an amplification reaction, the method comprising:
(A) treating a single strand template with a truncating oligonucleotide as a primer, along with nucleoside triphosphates and a polymerase under suitable conditions, such that the primer hybridizes to the template and an extension product of the primer is synthesized which is complementary to the template, wherein said truncating oligonucleotide can block replication of the primer-containing strand, thereby producing a truncated extension product;
(B) treating the sample under denaturing conditions to separate the extension product from its template;
(C) treating the extension product with an oligonucleotide or a truncating oligonucleotide as a primer, such that the primer hybridizes to the extension product and the truncated extension product referred to in step (A) is synthesized which is complementary to the extension product;
(D) optionally treating the sample under denaturing conditions to separate the extension product from the truncated extension product.

48. The method of claim 47, wherein the truncating oligonucleotide is a chimeric oligonucleotide.

49. The method of claim 47, wherein the target nucleic acid is DNA.

50. The method of claim 47, wherein the polymerase is a DNA polymerase.

51. The method of claim 50, wherein the DNA polymerase is Pfuturbo.

52. The method of claim 48, wherein the chimeric oligonucleotide comprises one or more 3′-5′ reversed-deoxynucleotides.

53. The method of claim 52, wherein the chimeric oligonucleotide comprises three 3′-5′ reversed-deoxynucleotides.

54. The method of claim 53, wherein the 3′-5′ reversed-deoxynucleotides are located about 6 to 8 nucleotides from the 3′ terminus of the oligonucleotide.

55. The method of claim 48, wherein the chimeric oligonucleotide comprises one or more 2′-OMe-ribonucleotides.

56. The method of claim 55, wherein the chimeric oligonucleotide comprises three 2′-OMe-ribonucleotides.

57. The method of claim 56, wherein the 2′-OMe-ribonucleotides are located about 6 to 8 nucleotides from the 3′ terminus of the oligonucleotide.

58. A kit for amplifying a target nucleic acid contained in a sample comprising:
one or more truncating oligonucleotides as primers, optionally an oligonucleotide as a primer, nucleoside triphosphates, a DNA polymerase, and suitable amplification reagents.

59. The kit of claim 58, wherein the one or more truncating oligonucleotides is a chimeric oligonucleotide.
